(19) United States
(12) Patent Application Publication
(10) Pub. No.: US 2013/0261165 A1
(43) Pub. Date: Oct. 3, 2013

(54) PREVENTION AND TREATMENT OF DISEASES CHARACTERIZED BY MESENCEPHALIC DOPAMINERGIC NEURON CELL DEATH

(71) Applicants: Ole Isacson, Cambridge, MA (US); Teresia Osborn, Belmont, MA (US)

(72) Inventors: Ole Isacson, Cambridge, MA (US); Teresia Osborn, Belmont, MA (US)

(73) Assignee: THE MCLEAN HOSPITAL CORPORATION, Belmont, MA (US)

(21) Appl. No.: 13/993,707

(22) PCT Filed: Dec. 16, 2011
(86) PCT No.: PCT/US11/65405

§ 371 Date: Jun. 13, 2013

Related U.S. Application Data
(60) Provisional application No. 61/423,964, filed on Dec. 16, 2010.

(51) Int. Cl. A61K 31/381

(52) U.S. Cl. CPC A61K 31/381
USPC 514/438

(57) ABSTRACT

Provided herein are methods for the prevention and treatment of diseases affecting mesencephalic dopaminergic neurons including, for example, Parkinson's disease. Suitable therapeutic agents for use in the methods described herein include, for example, agents that upregulate the expression En-1 and/or FoxA2 in target cells.

### CROSS-REFERENCE TO RELATED APPLICATIONS

This application claims benefit of U.S. Provisional Application 61/423,964, filed Dec. 16, 2010, which is hereby incorporated by reference in its entirety.

### FIELD OF THE INVENTION

The present invention relates to methods of prevention and treatment of diseases characterized by mesencephalic dopaminergic neuron apoptosis including, for example, Parkinson's disease.

### BACKGROUND OF THE INVENTION

The following discussion of the background of the invention is merely provided to aid the reader in understanding the invention and is not admitted to describe or constitute prior art to the present invention.

Parkinson's disease (PD) affects more than 500,000 patients in the U.S. alone. The cardinal clinical features of PD include (1) bradykinesia, (2) cogwheel rigidity, (3) resting tremor, and (4) postural instability. Dementia is a variable manifestation of PD, but occurs in approximately 40% of PD patients during the protracted course of the disease. Pathologically, the hallmark feature of PD is the degenerative loss of the dopaminergic (DA) neurons in the substantia nigra pars compacta (SNc), although the presence of extracellular melanin released from the degenerating neurons, reactive gliosis, and intracytoplasmic inclusions known as Lewy bodies in the remaining SNc neurons are also indicators. For a complete discussion, see Galvin et al., Synucleinopathies: Clinical and Pathological Implications, Arch. Neurol., Vol. 58, February 2001, which is hereby incorporated by reference in its entirety.

The nigral DA neurons of patients with PD exhibit several features. Pro-apoptotic genes are upregulated, and the proportion of neurons positive for activated caspase 3, a final effector of apoptosis, is five times higher than in healthy individuals. This suggests that nigral DA neurons of individuals with PD are more susceptible to apoptosis than are their healthy counterparts. See Sgado et al., Engrailed genes are self-autonomously required to prevent apoptosis in mesencephalic dopaminergic neurons, Development and Disease, Vol. 131(13), 3229, 2004, which is hereby incorporated by reference in its entirety.

### SUMMARY OF THE INVENTION

The present inventions are based on the discovery that increasing the expression of En-1 and/or FoxA2 is useful for prevention or treatment of diseases affecting mesencephalic dopaminergic neurons.

In one aspect, the invention provides a method for treating a disease affecting the midbrain dopaminergic (mDA) neurons of a patient (e.g., a human patient) comprising administering to a patient in need thereof a therapeutic composition comprising an agent that upregulates the biological activity of En-1 or FoxA2 in at least one neuronal cell type of said patient. In one embodiment, the therapeutic composition may be selected from the group consisting of: antidepressants (specifically SSRIs or NRIs), non-selective beta-adrenergic blockers, glutamate release inhibitors, phosphodiesterase 5 inhibitors, NSAID/COX inhibitors, antidiabetics, anticholinergics, topical corticosteroids, dopamine (D2) antagonists, intivirals, and ACE inhibitors.

In another embodiment, the invention provides a method for treating diseases affecting mesencephalic dopaminergic neurons in a patient (e.g., a human patient) by administering one or more therapeutic agents selected from the group consisting of: duloxetine, fluoxetine, citalopram, escitalopram, paroxetine, sertraline, atomoxetine, tomoxetine, mazindol, reboxetine, viloxazine, cyclobenzaprine, mesocarb, nefazodone, nefopam, sibutramine, tapentadol, tramadol, ziprasidone, pindolol, alprenolol, bucindolol, carteolol, carvedilol, labetalol, natolol, penbutolol, propranolol, sotalol, timolol, riluzole, vardenafil, vardenafil hydrochloride, avanafil, lodenafil, microdenafil, sildenafil citrate, tadalafil, udenafil, flufenamic acid, celecoxib, rofecoxib, etoricoxib, parecoxib, valdecoxib, mefenamic acid, meclofenamic acid, tolfenamic acid, acetohexamide, tolbutamide, tolazamide, chlorpropamide, glipizide, glyburide, glimepiride, gliclazide, methscopolamine bromide, dicyclomine, nortriptylene, desipramine, amitriptylene, flurandrenolide, fluocinolone, acetonide, hydrocortisone valerate, hydrocortisone butyrate, triamcinolone acetonide, mometasone furoate, sulpiride, haloperidol, domperidone, metoclopramide, amisulpride, tiapride, sultopride, pimozide, rimantidine hydrochloride, amantadine, zanamivir, oseltamivir, trandolapril, captopril, zofenopril, enalapril, ramipril, quinapril, perindopril, lisinopril, benazepril, fosinopril, megestrol acetate, methocarbamol, metoprolol tartrate, acebutolol hydrochloride, metaproterenol, methyldopa, moxalactam disodium, noscapine hydrochloride, oxyquinoline hemisulfate, phenelzine sulfate, methylprednisolone, nitromide, nylidrin hydrochloride, methicillin sodium, methylthiouracil, phenolphthalein, methimazole, naphazoline hydrochloride, norethindrone, orphenadrine citrate, pargyline hydrochloride, phenolbutazone, tolazoline hydrochloride, primidone, propylthiouracil, spironolactone, sulfapyridine, tetrahydrozoline hydrochloride, naltrexone hydrochloride, or esters or pharmaceutically acceptable salts thereof.

In some embodiments, the biological activity of En-1 and/or FoxA2 is upregulated at least 1.3 fold, 2.0 fold, 3.0 fold, 4.0 fold, 5.0 fold, 7.5 fold, 10 fold, or more. In further embodiments, the biological activity of En-1 and/or FoxA2 is assessed by measuring the biological activity of the En-1 or FoxA2 mRNA, respectively, or by measuring the biological activity of the En-1 or FoxA2 protein, respectively. In preferred embodiments, the biological activity of both En-1 and FoxA2 are increased.

In some embodiments, the disease affecting mesencephalic dopaminergic neurons is Parkinson's disease. In other embodiments, the neuronal cell type in which En-1 and/or FoxA2 is upregulated is a midbrain dopaminergic neuron including, for example, dopaminergic neurons in the A9 and/or A10 regions of the substantia nigra.

In another aspect, the invention provides an in vitro method of screening therapeutic agent candidates for therapeutic properties against a disease affecting mDA neurons, said method comprising: obtaining a sample of mammalian neurons; contacting neurons with a candidate compound; measuring the biological activity of the En-1 gene and/or the FoxA2 gene; and identifying the candidate compound as being useful for the treatment of a disease affecting mDA neurons when the biological activity of En-1 and/or FoxA2, respectively, is increased by at least 1.3 fold, 2.0 fold, 3.0 fold, 4.0 fold, 5.0 fold, 7.5 fold, 10 fold, or more. In other embodiments, the neurons may comprise mesencephalic neurons, and the mesencephalic neurons may comprise dopaminergic neurons. In still further embodiments, the measurement step further comprises measuring the amount of En-1 and/or FoxA2 mRNA and/or protein.

In still another aspect, the present invention provides a method of preventing or delaying the onset of a disease affecting mesenephalic dopaminergic neurons in a human suspected to be at risk for developing said disease comprising administering to said human a composition comprising duloxetine or a pharmaceutically acceptable salt thereof. The disease may, in some embodiments, be Parkinson's Disease. In further embodiments, the human may have a total UPDRS score selected from the group consisting of less than about 10, less than about 5, and 0. The average total UPDRS score of the patient may increase less than 0.15 units per week, less than 0.10 units per week, or less than 0.05 units per week, or no increase at all, after the initial symptomatic effect period of the administration of duloxetine or pharmaceutically acceptable salt of duloxetine. In still further embodiments, the pharmaceutically acceptable salt of duloxetine may be duloxetine HCl. In some embodiments, the human has not been diagnosed with Major Depressive Disorder. In other embodiments, the human is identified as having a loss of between 5% and 75% of midbrain dopaminergic neurons (e.g, neuron in the substantia nigra and/or specifically the A9 region of the substantia nigra). In specific embodiments, the human is identified as having lost at least about 5%, 10%, 15%, 20%, 25%, 30%, 35%, 40%, 45%, 50% 55% 60%, 65%, or 70 of the relevant dopaminergic neurons. In some embodiments, the percent of lost dopaminergic neurons is about not more than 55%, 60%, 65%, 70%, 75%, or 80%. Thus, the loss of the relevant midbrain dopaminergic neurons may be about, for example, 25%-70%, 35%-65%, 45%-65%, or about 55%-65%. The percent of lost dopaminergic neurons may be determined by any appropriate method including, for example, using positron emission technology (PET). In some embodiments, the therapeutically effective amount of duloxetine may be from about 10-120 mg/day, 20-90 mg/day, 30-80 mg/day or about 40-70 mg per day and administration may occur for about one week, one month, one year, two years, five years, or for the lifetime of the patient.

In still another aspect, the present invention provides a method of treatment of a patient suffering from a disease affecting mesenephalic dopaminergic neurons comprising administering to said patient a composition comprising duloxetine or a pharmaceutically acceptable salt thereof. The disease may, in some embodiments, be Parkinson's Disease. In further embodiments, the human may have a total UPDRS score selected from the group consisting of less than about 20, less than about 10, less than about 5, less than about 2, and 0. The average total UPDRS score of the patient may increase less than 0.15 units per week, less than 0.10 units per week, or less than 0.05 units per week, or no increase at all, after the initial symptomatic effect period of the administration of duloxetine or pharmaceutically acceptable salt of duloxetine. In still further embodiments, the pharmaceutically acceptable salt of duloxetine may be duloxetine HCl. In some embodiments, the human has not been diagnosed with Major Depressive Disorder. In other embodiments, the human is identified as having a loss of between 5% and 75% of midbrain dopaminergic neurons (e.g, neuron in the substantia nigra and/or specifically the A9 region of the substantia nigra). In specific embodiments, the human is identified as having lost at least about 5%, 10%, 15%, 20%, 25%, 30%, 35%, 40%, 45%, 50% 55% 60%, 65%, or 70 of the relevant dopaminergic neurons. In some embodiments, the percent of lost dopaminergic neurons is about not more than 55%, 60%, 65%, 70%, 75%, or 80%. Thus, the loss of the relevant midbrain dopaminergic neurons may be about, for example, 25%-70%, 35%-65%, 45%-65%, or about 55%-65%. The percent of lost dopaminergic neurons may be determined by any appropriate method including, for example, using positron emission technology (PET). In some embodiments, the therapeutically effective amount of duloxetine may be from about 10-120 mg/day, 20-90 mg/day, 30-80 mg/day or about 40-70 mg per day and administration may occur for about one week, one month, one year, two years, five years, or for the lifetime of the patient.

A compound (e.g., a candidate compound) that “upregulates the biological activity of En-1” refers to any compound that measurably increases any biological effect caused by an increase in the expression of the En-1 gene in that cell type (e.g., neuron). Upregulation of En-1 biological activity may be measured by increased amounts of RNA (e.g., mRNA) or En-1 protein. Alternatively, upregulated biological activity may be measured functionally including, for example, by observing increased rates of axonal migration and/or stem cell differentiation.

A compound (e.g., a candidate compound) that “upregulates the biological activity of FoxA2” refers to any compound that measurably increases any biological effect caused by an increase in the expression of the FoxA2 gene in that cell type (e.g., neuron). Upregulation of FoxA2 biological activity may be measured by increased amounts of RNA (e.g., mRNA) or FoxA2 protein. Alternatively, upregulated biological activity may be measured functionally including, for example, by observing increased rates of stem cell differentiation or other developmental alterations associated with FoxA2.

By “treating” is meant the medical management of a patient with the intent that a cure, amelioration, or prevention of a disease affecting mDA neurons will result. This term includes active treatment, that is, treatment directed specifically toward improvement of a disease affecting mDA neurons, and also includes causal treatment, that is, treatment directed toward removal of the cause of the disease, pathological condition, or disorder. In addition, this term includes palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. The term “treating” also includes symptomatic treatment, that is, treatment directed toward constitutional symptoms of the disease.

By “an amount sufficient” is meant the amount of a compound, alone or in combination with another therapeutic regimen, required to treat, prevent, or reduce a disease affecting mDA neurons such as PD in a clinically relevant manner. An sufficient amount of an active compound used to practice the present invention for therapeutic treatment of conditions affecting mDA neurons varies depending upon the manner of administration, the age, body weight, and general health of the patient.

By “delaying the onset of” symptoms associated with a disease affecting mDA neurons is meant either (A) a reduction in the rate of mDA neuron loss as compared to that which would be experienced by an untreated patient, or (B) a reduction in the rate of increase of UPDRS score as compared to that which would be experienced by an untreated patient. As used herein, the rate of mDA neuron loss is measured as a percentage lost, and the rate of increase of UPDRS score is measured in points per week.

As used herein, a Unified Parkinson's Disease Rating Scale (UPDRS) score is a metric used in evaluation by a clinician of motor abilities or impairment of PD patients. The UPDRS was recently reviewed and updated, and is regarded as a standard mean to evaluate PD patients (see Movement Disorders, Vol. 22, No. 1, 2007, pp. 41-47; Movement Disorder Society-Sponsored Revision of the Unified Parkinson's Disease Rating Scale (MDS-UPDRS): Process, Format, and Clinimetric Testing Plan, by Dr. Christopher G. Goetz et al, doi: 10.1002/mds.21198).

As used herein, “mesencephalic dopaminergic neurons” refers to neuron cells that develop from the mesencephalon and utilize dopamine as a neurotransmitter.

As used herein, “patient” refers to a mammal (e.g., human) that has been diagnosed with a disease affecting mDA neurons or identified as having an increased likelihood of developing a disease affecting mDA neurons, or a mammal (e.g., human) who is suspected of having an increased likelihood of developing a disease affecting mDA neurons.

### BRIEF DESCRIPTION OF THE FIGURES

FIG. 1 is schematic illustration showing the timeline for cell culture and treatment with the candidate compounds.

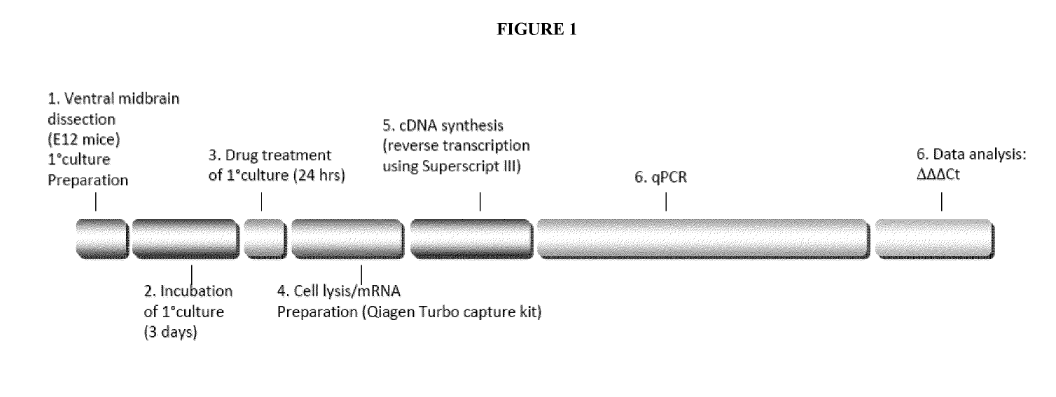

FIG. 2 is a pair of bar graphs illustrating the fold increase in expression of (A) En-1 and (B) FoxA2 genes upon treatment with either duloxetine HCl or saline vehicle. Adult mice were treated with duloxetine HCl (20 mg/kg/day) by subcutaneous injection for 12 days (n=8). En-1 mRNA expression was increased (p=0.0064) in the substantia nigra of treated mice (1.98±0.098) versus control (1.08±0.23). FoxA2 mRNA expression was also increased (p=0.0026) in the substantia nigra of treated mice (2.19±0.23) versus control (1.02±0.21). Data were obtained by qPCR analysis using GAPDH and β-actin as housekeeping genes followed by ΔΔCt calculation.

FIG. 3 is a bar graph illustrating the percent DA neuron survival as a percentage of the control after pre-treatment with duloxetine HCl (10 μM) or vehicle control 24 hours prior to 6-OHDA treatment (1-50 μM). The data demonstrate that duloxetine pre-treatment reduces 6-OHDA-induced toxicity in these neurons. Survival of duloxetine-treated cultures was 107%±7 compared to 74%±18 for vehicle control.

### DETAILED DESCRIPTION

The present invention relates generally to methods for prevention or treatment of diseases affecting mDA neurons, including diseases that are characterized by a loss of mDA neurons. Such diseases may be treated by administering to a patient in need thereof, any one or more of the therapeutic agents (or therapeutic agents from the classes of therapeutic agents) disclosed herein. Preferably, the therapeutic agents increase the expression of En-1 and/or FoxA2 in the mDA neurons of the patient. More preferably, the therapeutic agents prevent or delay mDA neuron loss and/or onset of symptoms.

The engrailed gene (“En-1”) encodes a homeodomain transcription factor known to be involved in the embryonic development of many organisms (e.g., humans) with activity in on variety of developing tissues and cell types, including the dopaminergic neurons of the brain. In particular, it has been discovered that En-1 facilitates axonal guidance during neuronal development from stem cells. Cord et al., Mol. Cell. Neurosci. 45: 324-34, 2010.

In mammals, two homologues of En have been identified, En1 and En2. They are both expressed by all mDA neurons from early in development into the adult. Homologous recombinant mutant mice with both En1 and En2 knockouts show a large deletion in the midbrain and anterior hindbrain. Despite this deficiency, the mDA neurons are generated, become postmitotic and express tyrosine hydroxylase (TH), the rate-limiting enzyme of dopamine synthesis. However, soon thereafter, the cells disappear, and by PO the entire mDA system is absent. Sgado et al., Development and Disease, 131 (13) 2004.

En-1 has also been shown to increase mitochondrial complex I activity and protect mDA neurons against neurotoxicity. Mitochondrial impairment, in turn, is a hallmark of PD. First, mitochondrial complex I activity is reduced in the brains of people with the disease. Second, MPTP selectively kills mDA neurons through inhibition of complex I. Third, genetic studies in familial forms of PD implicate mitochondrial dysfunctions, as mutated nuclear genes encoding PTEN-induced putative kinase, Parkin, alpha-synuclein, DJ-1, and LRRK2 all directly or indirectly affect mitochondrial physiology. For a complete discussion of the connection between En-1 activity and PD, see, e.g., Alvarez-Fischer et al., Engrailed protects mouse midbrain dopaminergic neurons against mitochondrial complex I insults, Nature Neuroscience, Vol. 14, No. 10, 1260-1268, October 2011.

The FoxA2 gene (also known as hepatocyte nuclear factor 3-beta) is a forkhead transcription factor which is also known to play a role in the development of dopaminergic neurons from stem cells. Nakatani et al. Dev. Biol. 339: 101-113, 2010. FoxA2 is an essential transcription factor involved in midbrain development and phenotype. As such, it is a master regulator of the enzymes associated with the dopaminergic phenotype in the midbrain. Haplo-insufficiency of FoxA2 shows an accelerated form of aging of the mDA neurons that is very reminiscent, both in its style of degeneration and the loss of DA neurons most vulnerable to Parkinson's disease.

Furthermore, FoxA2 has been shown to regulate the response of dopamine neurons to oxidative stress, which is considered to be a primary cause of PD. See, e.g., Kittappa et al., The FoxA2 Gene Controls the Birth and Spontaneous Degeneration of Dopamine Neurons in Old Age, PLoS Biology, Volume 5:12, 2875-2884, December 2007. Kittappa et al. describe that FoxA2 heterozygous mice spontaneously develop significant motor problems late in life, and an associated late-onset degeneration of dopamine neurons, which asymmetrically and preferentially affects dopamine neurons of the SN, while leaving the VTA intact, the exact pattern of sensitivity seen in PD patients.

### Measurement of PD Symptoms

PD is a progressive neurodegenerative disorder characterized by dopaminergic neuronal loss in the substantia nigra and the presence of cytoplasmic inclusions (Lewy bodies) in surviving neurons. The classical clinical features of PD include progressive tremor, rigidity, and bradykinesia.

The symptoms of PD in patients are frequently expressed as Unified Parkinson's Disease Rating Scale (UPDRS) score. Most frequently, so-called “part 3” is used in evaluation by a clinician of motor abilities/impairment of PD patients. The UPDRS was recently reviewed and updated, and is regarded as a standard mean to evaluate PD patients (see Movement Disorders, Vol. 22, No. 1, 2007, pp. 41-47; Movement Disorder Society-Sponsored Revision of the Unified Parkinson's Disease Rating Scale (MDS-UPDRS): Process, Format, and Clinimetric Testing Plan, by Dr. Christopher G. Goetz et al, doi: 10.1002/mds.21198). While no generally accepted cutoff scores are recognized as unequivocally indicating that a patient is suffering from PD, a score of UPDRS score of greater than 20 is used herein to indicate the presence of PD.

The Total UPDRS (Unified Parkinson's Disease Rating Scale) score represents the level or severity of Parkinson's disease symptoms. It is used for measuring the change from baseline in efficacy variables during the treatment. UPDRS consists of a three-part test. A total of 31 items are included in Parts I, II and III test. Each item receives a score ranging from 0 to 4 where 0 represents the absence of impairment and 4 represents the highest degree of impairment. The sum of Parts I, II and III at each study visit provides a total UPDRS score. Part I is designed to rate mentation, behavior and mood (items 1-4). It is collected as historical information. Part II (items 5-17) is also historical information. Part III (items 18-31) is a motor examination at the time of a visit. The scoring is conducted as follows:

### Part I: Mentation, Behavior and Mood

Item 1. Intellectual Impairment

0: None.

Mild—Consistent forgetfulness with partial recollection of events and no other difficulties.

2: Moderate memory loss, with disorientation and moderate difficulty in handling complex problems. Mild but definitive impairment of function at home with need of occasional prompting.

3: Severe memory loss with disorientation for time and often to place. Severe impairment in handling problems.

4: Severe memory loss with orientation preserved to person only. Unable to make judgments or solve problems. Requires much help with personal care; cannot be left alone at all.

Item 2. Thought Disorders (Due to Dementia or Drug Intoxication)

0: None.

1: Vivid dreaming.

2: Benign hallucinations with insight retained.

3: Occasional to frequent hallucinations or delusions; without insight; could interfere with daily activities.

4: Persistent hallucinations, delusions or florid psychosis. Not able to care for self Item 3. Depression

0: Not present.

1: Periods of sadness or guilt greater than normal. Never sustained for days or weeks.

2: Sustained depression (1 week or more)

3: Sustained depression with vegetative symptoms (insonmia, anorexia, weight loss, loss of interest).

4: Sustained depression with vegetative symptoms and suicidal, thoughts or intent.

Item 4. Motivation/Initiative

0: Normal.

1: Less assertive than usual; more passive.

2: Loss of initiative or disinterest in elective (nonroutine) activities.

3: Loss of initiative or disinterest in day-to-day (routine) activities.

4: Withdrawn, complete loss of motivation.

### Part II: Activities of Daily Living (Score 0-4)

Item 5. Speech

0: Normal.

1: Mildly affected. No difficulty being understood.

2: Moderately affected. Sometimes asked to repeat statements.

3: Severely affected. Frequently asked to repeat statements.

4: Unintelligible most of the time.

Item 6. Salivation

0: Normal.

1: Slight but definite excess of saliva in mouth; may have nighttime drooling.

2: Moderately excessive of saliva; may have minimal drooling.

3: Marked excess of saliva with some drooling.

4: Marked drooling, requires constant tissue or handkerchief

Item 7: Swallowing

0: Normal.

1: Rare choking

2: Occasional choking

3: Requires soft food.

4: Requires nasogastric tube or gastrotomy feeding.

Item 8. Handwriting

0: Normal.

1: Slightly slow or small.

2: Moderately slow or small; all words are legible.

3: Severely affected; not all words are legible.

4: The majority of words are not legible.

Item 9. Cutting Food, Handling Utensils

0: Normal.

1: Somewhat slow, but no help needed.

2: Can cut most foods, although clumsy and slow; some help needed.

3: Food must be cut by someone, but can still feed slowly.

4: Needs to be fed.

Item 10. Dressing

0: Normal.

1: Somewhat slow, but no help needed.

2: Occasional assistance with buttoning, getting arms in sleeves.

3: Considerable help required, but can do some things alone.

4: Helpless.

Item 11. Hygiene

0: Normal.

1: Somewhat slow, but no help needed.

2: Needs help to shower or bathe, or very slow in hygienic care.

3: Requires assistance for washing, brushing teeth, combing hair, going to bathroom.

4: Foley catheter or other mechanical aids.

Item 12. Turning in Bed and Adjusting Bed Clothes

0: Normal.

1: Somewhat slow and clumsy, but no help needed.

2: Can turn alone or adjust sheets, but with great difficulty.

3: Can initiate, but not turn or adjust sheets alone.

4: Helpless.

Item 13. Falling (Unrelated to Freezing)

0: None.

1: Rare falling.

2: Occasionally falls, less than once per day.

3: Falls an average of once daily.

4: Falls more than once daily.

Item 14. Freezing when Walking

0: None.

1: Rare freezing when walking; may have starthesitation.

2: Occasional freezing when walking

3: Frequent freezing. Occasionally falls from freezing.

4: Frequent falls from freezing.

Item 15. Walking

0: Normal.

1: Mild difficulty. May not swing arms or may tend to drag leg.

2: Moderate difficulty, but requires little or no assistance.

3: Severe disturbance of walking, requiring assistance.

4: Cannot walk at all, even with assistance.

Item 16. Tremor

0: Absent.

1: Slight and infrequently present.

2: Moderate; bothersome to patient.

3: Severe; interferes with many activities.

4: Marked; interferes with most activities.

Item 17. Sensory Complaints Related to Parkinsonism

0: None.

1: Occasionally has numbness, tingling or mild aching

2: Frequently has numbness, tingling or aching; not distressing.

3: Frequent painful sensations.

4: Excruciating pain.

### Part III: Motor Examination (Score 0-4)

Item 18. Speech

0: Normal.

1: Slight loss of expression, diction and/or volume.

2: Monotone, slurred but understandable; moderately impaired.

3: Marked impairment, difficult to understand.

4: Unintelligible.

Item 19. Facial Expression

0: Normal.

1: Minimal hypomimia, could be normal “Poker Face”.

2: Slight but definitely abnormal diminution of facial expression.

3: Moderate hypomania; lips parted some of the time.

4: Masked or fixed faces with severe or complete loss of facial expression; lips parted ½ inch or more.

Item 20. Tremor at Rest

a) Face, lips and chin; b) Right hand; c) Left hand; d) Right foot; e) Left foot 0: Absent.

1: Slight and infrequently present.

2: Mild in amplitude and persistent; or moderate in amplitude, but only intermittently present.

3: Moderate in amplitude and present most of the time.

4: Marked in amplitude and present most of the time.

Item 21. Action or Postural Tremor of Hands

0: Absent.

1: Slight; present with action.

2: Moderate in amplitude, present with action.

3: Moderate in amplitude with posture holding as well as action.

4: Marked in amplitude; interfere with feeding.

Item 22. Rigidity (Judged on Passive Movement of Major Joints with Subject Relaxed In Sitting Position. Cogwheeling to be Ignored) a) neck; b) right upper extremities; c) left upper extremities; d) right lower extremities; e) left lower extremities

0: Absent.

1: Slight or detectable only when activated by mirror or other movements.

2: Mild or moderate.

3: Marked, but full range of motion easily achieved.

4: Severe, range of motion achieved with difficulty.

Item 23. Finger Taps (Subject Taps Thumb with Index Finger in Rapid succession with widest amplitude possible, Each Hand Separately) a) Right hand; b) Left hand

0: Normal>15/5 sec.

1: Mild slowing and/or reduction in amplitude (1114.5 sec).

2: Moderately impaired. Definite and early fatiguing. May have occasional arrests in movement (7-10/5 sec).

3: Severely impaired. Frequent hesitation in initiating movements or arrests in ongoing movement (3-6/5 sec).

4: Can barely perform the task (0-2/5 sec).

Item 24. Hand Movement (Subject Opens and Closes Hands in Rapid Succession with Widest Amplitude Possible, Each Hand Separately) a) Right hand; b) Left hand

0: Normal.

1: Mild slowing and/or reduction in amplitude.

2: Moderately impaired. Definite and early fatiguing. May have occasional arrests in movements.

3: Severely impaired. Frequent hesitation in initiating movements or arrests in ongoing movement.

4: Can barely perform the task.

Item 25. Rapid Alternating Movements of Hands (Pronation, Supination Movements of Hands, Vertically or Horizontally with as Large an Amplitude as Possible, Both Hands Simultaneously)

0: Normal.

1: Mild slowing and/or reduction in amplitude.

2: Moderately impaired. Definite and early fatiguing. May have occasional arrests in movement.

3: Severely impaired. Frequent hesitation in initiating movements or arrests in ongoing movement.

4: Can barely perform the task.

Item 26. Leg Agility (Subject Taps Heel on Ground in Rapid Succession, Picking Up Entire Leg. Amplitude should be about 3 Inches)

0: Normal.

1: Mild slowing and/or reduction in amplitude.

2: Moderately impaired. Definite and early fatiguing. May have occasional arrest in movement.

3: Severely impaired. Frequent hesitation in initiating movements or arrests in ongoing movement.

4: Can barely perform the task.

Item 27. Arising from Chair (Subject Attempts to Arise from a Straight-Back Wood or Metal Chair with Arms Folded Across)

0: Normal.

1: Slow, or may need more than one attempt.

2: Pushes selfup from arms of seat.

3: Tends to fall back and may have to try more than one time, but can get up without help.

4: Unable to arise without help.

Item 28. Posture

0: Normal erect.

I: Not quite erect, slightly stooped posture; could be normal for older person.

2: Moderately stooped posture, definitely abnormal, can be slightly leaning to one side.

3: Severely stooped posture with kyphosis; can be moderately leaning to one side.

4: Marked flexion with extreme abnormality of posture.

Item 29. Gait 0: Normal.

1: Walks slowly, may shuffle with short steps, but no festination or propulsion.

2: Walks with difficulty, but requires little or no assistance; may have some festination, short steps, or propulsion.

3: Severe disturbance, of gait requiring assistance.

4: Cannot walk at all, even with assistance.

Item 30. Postural Stability (Response to Sudden Posterior Displacement)

0: Normal.

1: Retropulsion, but recovers unaided.

2: Absence of postural response; would fall if not caught by examiner.

3: Very unstable, tends to lose balance spontaneously.

4: Unable to stand without assistance.

Item 31. Body Bradykinesia and Hypokinesia (Combining Slowness, Hesitancy, Decreased Arm Swing, Small Amplitudes and Poverty of Movement in General)

0: None.

1: Minimal slowness, giving movement a deliberate character; could be normal for some person. Possibly reduced amplitude.

2: Mild degree of slowness and poverty of movement which is definitely abnormal. Alternatively, some reduced amplitude.

3: Moderate slowness, poverty or small amplitude of movement.

4: Marked slowness, poverty or small amplitude of movement.

For a therapy to be effective in modifying PD, neuroprotection must be introduced as early in the course of disease as possible. This is due to the reason that by the time a diagnosis of PD is made, 50% to 80% of nigral cell loss usually has already occurred (Simpins N, Jankovic J. Neuroprotection in Parkinson Disease. Arch Intern Med, Jul. 28, 2003, Vol 163: 1650-1654). Therefore, there is a need for protective measures that may be introduced before a definitive diagnosis has been made (i.e., before a UPDRS score of greater than 20 has been observed).

### Measuring mDA Neuron Loss

In addition to quantifying the symptoms of PD through calculation of a UPDRS score, the loss of mDA neurons may also be quantified as a means of determining the presence, absence, or progression of PD. Such a quantification may be achieved through any suitable method. One such method is Positron emission tomography (PET), a nuclear medicine imaging technique that produces a three-dimensional image or picture of functional processes in the body. The system detects pairs of gamma rays emitted indirectly by a positron-emitting radionuclide (tracer), which is introduced into the body on a biologically active molecule. Three-dimensional images of tracer concentration within the body are then constructed by computer analysis. In modern scanners, three dimensional imaging is often accomplished with the aid of a CT X-ray scan.

Radionuclides used in PET scanning are typically isotopes with short half-lives such as carbon-11 (˜20 min), nitrogen-13 (˜10 min), oxygen-15 (˜2 min), and fluorine-18 (˜110 min). These radionuclides are incorporated either into compounds normally used by the body such as glucose (or glucose analogues), water, or ammonia, or into molecules that bind to receptors or other sites of drug action. Such labeled compounds are known as radiotracers. It is important to recognize that PET technology can be used to trace the biologic pathway of any compound in living humans (and many other species as well), provided it can be radiolabeled with a PET isotope. Thus, the specific processes that can be probed with PET are virtually limitless, and radiotracers for new target molecules and processes are continuing to be synthesized; as of this writing there are already dozens in clinical use and hundreds applied in research. At present, however, by far the most commonly used radiotracer in clinical PET scanning is fluorodeoxyglucose, an analogue of glucose that is labeled with fluorine-18.

Due to the short half-lives of most positron-emitting radioisotopes, the radiotracers have traditionally been produced using a cyclotron in close proximity to the PET imaging facility. The half-life of fluorine-18 is long enough that radiotracers labeled with fluorine-18 can be manufactured commercially at offsite locations and shipped to imaging centers. Recently rubidium-82 generators have become commercially available. These contain strontium-82 which decays by electron capture to positron emitting rubidium-82. Preferred ligands for use in measuring mDA loss include CFT and C-CIT.

### Duloxetine

Duloxetine is a selective serotonin and norepinephrine reuptake inhibitor (SSNRI) for oral administration. Eli Lilly markets duloxetine in the United States under the trade name Cymbalta® as a delayed release capsule containing enteric-coated pellets of the duloxetine. It is indicated for the treatment of major depressive disorder and for the treatment of diabetic peripheral neuropathic pain. These enteric-coated pellets are designed to prevent degradation of the drug in the acidic environment of the stomach.

Duloxetine is acid labile and acid hydrolysis of its ether linkage results in a thienyl alcohol and I-naphthol. 50% of the dosage is hydrolyzed to I-naphthol within one hour at a pH of 1.0, which is achieved under fasting conditions. At a pH of 2.0, 10% of the dosage degrades to I-naphthol in one hour and at a pH of 4.0, 10% degradation would take up to 63 hours. Typically, such acid sensitive compounds are formulated as enteric-coated pellets to protect them from degradation. Typical daily dosage amounts range from about 40 to 60 milligrams once daily, or 20 to 30 milligrams twice daily.

A pharmaceutically acceptable salt of duloxetine may also be used in the methods disclosed herein. The pharmaceutically acceptable salt may be selected from the group consisting of sodium, potassium, lithium, ammonium, calcium, magnesium salts, salts of primary, secondary or tertiary amines, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, diecycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, and mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, and heterocyclic, or may be derived from hydrochloric acid, hydrobromic acid, sulfuric acid, nitric acid, phosphoric acid, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, or salicylic acid. In a preferred embodiment, the pharmaceutically acceptable salt may be derived from HCl.

### Formulation and Administration of Therapeutic Agents

Therapeutic agents of the invention can be administered to a patient, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York.

Pharmaceutical formulations of a therapeutically effective amount of a peptide agent or candidate compound of the invention, or pharmaceutically acceptable salt-thereof, can be administered orally, parenterally (e.g. intramuscular, intraperitoneal, intravenous, or subcutaneous injection), or by intrathecal or intracerebroventricular injection in an admixture with a pharmaceutically acceptable carrier adapted for the route of administration.

Methods well known in the art for making formulations are found, for example, in Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The amount of active ingredient in the compositions of the invention can be varied. One skilled in the art will appreciate that the exact individual dosages may be adjusted somewhat depending upon a variety of factors, including the protein being administered, the time of administration, the route of administration, the nature of the formulation, the rate of excretion, the nature of the subject's conditions, and the age, weight, health, and gender of the patient. Generally, dosage levels of between 0.1 mg/kg to 100 mg/kg of body weight are administered daily as a single dose or divided into multiple doses. Desirably, the general dosage range is between 250 mg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

If more than one agent is employed, each agent may be formulated in a variety of ways that are known in the art. Desirably, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. The individually or separately formulated agents can be packaged together or separately, or may be co-formulated.

Generally, when administered to a patient, the timing dosage of any of the therapeutic agent(s) will depend on the nature of the agent, and can readily be determined by one skilled in the art. Each agent may be administered once or repeatedly over a period of time (e.g., including for the entire lifetime of the patient).

### EXAMPLES

The present methods, thus generally described, will be understood more readily by reference to the following examples, which are provided by way of illustration and are not intended to be limiting of the present methods and kits.

### Example 1

### Identification of Therapeutic Compounds that Increase En-1 And/or FoxA2 Expression for the Prevention and Treatment of Diseases Affecting mDA Neurons

One library consisting of FDA-approved and non-FDA approved drugs (NINDS) was screened for drugs with an ability to up-regulate En-1 and/or FoxA2 expression in neuronal cells. Candidate compounds were screened in a gene expression assay using a qPCR detection methodology in a primary ventral midbrain preparation of E12 mice. In the initial screening, several therapeutic candidates were identified as those that increased both En-1 and FoxA2 expression by at least 1.3-fold.

To determine the extent of upregulation in the compounds of interest after initial screening, primary cultures of ventral midbrain cells of E12 CD1 mice were prepared. The embryonic ventral midbrains were removed and stored in HBSS on ice. The dissection time was less than 30-60 minutes. The ventral midbrain tissue was dissociated by removal of the HBSS and addition of 1 ml of 0.05% trypsin-EDTA for 10 minutes at 37° C. During incubation, the tissue and the trypsin-EDTA solution was mixed by occasional careful swirling of the tube. The reaction was stopped, the trypsin aspirated, and 1 ml of 50% HBSS/FBS was added. The HBSS/FBS was then aspirated and tissue washed first in 1 ml HBSS and then in 1 ml E12 media (Dulbecco's modified Eagle's medium/F12 (Invitrogen) containing 5% fetal bovine serum, 1×N2 supplement A (Stem Cell Technologies), glucose (0.36%, Sigma), bovine serum albumin (0.25%, Invitrogen) and penicillin-streptomycin (Invitrogen). The tissue is triturated using an autoclaved glass pasteur pipette with narrowed opening and a stripettor. The cell suspension was centrifuged at 1000 rpm for 5 min, the supernatant aspirated, and the cells resuspended in E12 media. One hundred thousand cells per well were plated in 96 well PLO/laminin coated plates and incubated for 3 days without candidate compound treatment. Following this initial culture, cells were exposed to the various drugs, individually, at 10 μM for 24 hours. Candidate compound treatment was stopped by cell lysis. The mRNA was isolated and cDNA was synthesized using the Qiagen Turbocapture™ kit (Qiagen Inc., Valencia, Calif.) using Superscript III® reverse transcriptase (Life Technologies, Carlsbad, Calif.) according to manufacturer's protocol. En-1 and FOXA2 mRNA in the cultured cells was quantified using qPCR. For quantification, the cDNA was mixed with TaqMan assay primers for 18S rRNA (Applied Biosystems, Pre-Developed Assay Reagents, 20×; catalog#4333760F), FoxA2 (Applied Biosystems, assayID Mm00438709_ml) or En-1 (Applied Biosystems, assayID Mm00839704_mH), TaqMan Universal PCR Master Mix (Applied Biosystems), and nuclease free water. Assay fluorescence was determined using a Step-One Plus fluorescence plate reader in 96-well plates at a standard reaction time for ramp speed. Individual wells/treatment conditions result in Ct values>36 were omitted from the analysis.

The fold change in mRNA expression level was calculated according to the following formulas:

Fold change=2^{-ΔΔCt} wherein [1]

ΔΔCt=(Ct_{target gene}−Ct_{18S})_{treated}−(Ct_{target gene}−Ct_{18S})_{untreated}. [2]

In formula 2, Ct_{target gene} refers to the Ct value determined for either En-1 or FOXA2 (as appropriate) and Ct_{18S} refers to the Ct value determined for the mRNA encoding the 18S ribosomal subunit, each measured by qPCR.

Table 1 illustrates the results for the induction of En-1 and FoxA2 expression by a series of therapeutic agents. The degree of induction of En-1 and FoxA2 expression is calculated for antihypertensives, antidepressants (including SSRIs and NRIs), anti-ALS/glutamate release inhibitors, phosphodiesterase 5 inhibitors, NSAID/COX inhibitors, antidiabetics, anticholinergics, corticosteroids, antipsychotics (dopamine (D2) antagonists), antivirals, and antihypertensives (ACE inhibitors). The most active of these include duloxetine HCL, pindolol, riluzole, vardenafil HCL, flufenamic acid, acetohexamide, methscopolamine bromide, flurandrenolide, sulpiride, rimantadine HCL, and trandolapril.

The results of the library screening identified the following therapeutic agents as having a significant inducing effect on En-1 and/or FoxA2 expression in primary ventral midbrain cultures and are useful for treating Parkinson's Disease.

TABLE 1

| | Fold Increase | Fold Increase | |
|---|---|---|---|
| | in En-1 | in FoxA2 | |
| Therapeutic Agent | Expression | Expression | Comments |
| Duloxetine | 19.0 | 49.5 | Antidepressant-SSRI and NRI |
| hydrochloride | | | |
| Pindolol | 9.8 | 44.1 | Antihypertensive-non-selective beta- |
| | | | adrenergic blocker, CNS available |
| Riluzole | 6.2 | 25.9 | Anti-ALS-glutamate release inhibitor |
| Vardenafil | 1.4 | 19.1 | Erectile dysfunction agent-phosphodiesterase |
| hydrochloride | | | 5 inhibitor |
| Flufenamic acid | 2.6 | 3.4 | NSAID-COX inhibitor |
| Acetohexamide | 2.3 | 3.0 | Antidiabetic-hypoglycemic, sulfonylurea |
| Methscopolamine | 1.9 | 1.9 | Anti-peptic ulcer disease-anticholinergic effect |
| bromide | | | |
| Flurandrenolide | 1.5 | 1.9 | Topical corticosteroid |
| Sulpiride | 1.4 | 1.7 | Antipsychotic, antidepressant (Dopamine (D2) |
| | | | antagonist) |
| Rimantadine | 1.3 | 2.7 | Antiviral-influenza treatment and profylax, |
| hydrochloride | | | also possible motor benefits in PD patients |
| Trandolapril | 17.9 | Not | Antihypertensive-ACE inhibitor |
| | | Determined | |
| Megestrol acetate | 2.14 | 1.89 | Endocrine-metabolic agent |
| Methocarbamol | 1.83 | 2.30 | Skeletal muscle relaxant, centrally acting |
| Metoprolol tartrate | 2.18 | 2.21 | Antianginal, antiarrhythmic, antihypertensive, |
| | | | Beta-adrenergic blocker, cardioselective |
| Acebutolol | 0.80 | 1.28 | Antianginal, antiarrhythmic, antihypertensive, |
| hydrochloride | | | Beta-adrenergic blocker, cardioselective |
| Metaproterenol | −0.09 | 1.74 | Beta-2 adrenergic agonist, bronchodilator |
| Methyldopa | −1.41 | 2.47 | Antihypertensive, alpha-adrenergic agonist |
| Moxalactam | 4.34 | 7.31 | Antibacterial/cephalosporin |
| disodium | | | |
| Noscapine | 1.83 | 1.45 | Cough suppressant |
| hydrochloride | | | |
| Oxyquinoline | 0.54 | 1.75 | antiprotozoal |
| hemisulfate | | | |
| Phenelzine sulfate | −0.69 | 2.50 | Antidepressant, monoamine oxidase inhibitor, |
| | | | nonselective |
| Methylprednisolone | 1.38 | 1.41 | Adrenal glucocorticoid, endocrine-metabolic |
| | | | agent, immune suppressant |
| Nitromide | 0.28 | 1.55 | antiprotozoal |
| Nylidrin | −0.66 | 1.70 | Peripheral vasodilator |
| hydrochloride | | | |
| Methicillin sodium | 1.29 | 2.46 | Penicillin, pericillinase-resistant |
| Methylthiouracil | −0.19 | 2.34 | Antithyroid agent |
| Phenolphthalein | 6.63 | 3.22 | Laxative, stimulant |
| Methimazole | 0.20 | 1.97 | Antithyroid agent, thionamide |
| Naphazoline | 8.95 | 1.99 | Alpha-adrenergic agonist, decongestant, |
| hydrochloride | | | imidazoline, sympathomimetic |
| Norethindrone | 3.03 | Not | Contraceptive, progestin, endocrine-metabolic agent |
| | | Determined | |
| Orphenadrine | 1.69 | 1.82 | Antimuscarinic, skeletal muscle relaxant, |
| citrate | | | centrally acting |
| Pargyline | 1.73 | 1.27 | Selective inhibitor of MAO-B |
| hydrochloride | | | |
| Phenolbutazone | 1.82 | 2.21 | NSAID, Analgesic, Antigout, Antirheumatic, |
| | | | Central Nervous System Agent, Pyrazolone |
| Tolazoline | 2.51 | 2.67 | Alpha-adrenergic blocker, peripheral |
| hydrochloride | | | vasodilator |
| Primidone | 0.86 | 1.43 | Anticonvulsant, barbiturate, intermediate-acting |
| Propylthiouracil | 1.30 | 1.68 | Antithyroid agent, thionamide |
| Spironolactone | 1.34 | 1.74 | Aldosterone receptor antagonist, antiandrogen, |
| | | | diuretic, potassium sparing, cardiovascular agent |
| Sulfapyridine | 1.48 | 1.56 | sulfonamide |
| Tetrahydrozoline | 1.06 | 2.06 | Decongestant, imidazoline |
| hydrochloride | | | |
| Tolbutamide | 21.64 | 34.99 | First-generation sulfonylurea, diagnostic agent, |
| | | | pancreatic function, hypoglycemic |
| Naltrexone | 2.09 | 3.39 | Ethanol dependency, opioid antagonist, opioid |
| hydrochloride | | | dependency, toxicology-antidote agent |

### Example 2

### Use of Duloxetine to Increase En-1 and/or FoxA2 Expression For the Prevention and Treatment of Diseases Affecting mDA Neurons

Duloxetine HCl (20 mg/kg/day) or saline vehicle was administrated subcutaneously into adult male C57 mice over a period of 12 days. 24 hours after the last injection, the animals were sacrificed and perfused in heparinized saline. Substantia nigra pars compacta was fresh dissected and snap frozen in RNA preserving solution in liquid nitrogen. Tissue lysates and mRNA were prepared using RNeasy Lipid kit (product number 74804, Qiagen) and cDNA was synthesized using Superscript III first strand synthesis kit (product number 18080-044, Invitrogen). Changes in mRNA expression due to duloxetine HCl treatment were determined by quantitative PCR using a Step One Plus Instrument and TaqMan® reagents (Applied Biosystems). For quantification, the cDNA was mixed with TaqMan assay primers for Beta actin (Applied Biosystems, Pre-Developed Assay Reagents, 20×; catalog#4352932E), GAPD (Applied Biosystems, Pre-Developed Assay Reagents, 20×; catalog#4352933E) FoxA2 (Applied Biosystems, assayID Mm00438709 ml) or En-1 (Applied Biosystems, assayID Mm00839704_mH), TaqMan Universal PCR Master Mix (Applied Biosystems), and nuclease free water. Assay fluorescence was determined using a Step-One Plus fluorescence plate reader (Applied Biosystems) in 96-well plates at a standard reaction time for ramp speed. Beta actin and GAPD were used as housekeeping genes and fold regulation was calculated using the delta Ct method. Individual wells/treatment conditions with Ct values>36 were omitted from the analysis.

The fold change in mRNA expression level was calculated according to formula 1 and formula 2:

Fold change=2^{-ΔΔCt} [1]

wherein

ΔΔCt=(Ct_{target gene}−Ct_{housekeeping gene})_{treated}−(Ct_{target gene}−Ct_{housekeeping gene})_{untreated} [2]

Ct_{target gene} refers to the Ct value determined for either En-1 or FOXA2 (as appropriate) and Ct_{housekeeping gene} refers to the Ct value determined for the mRNA encoding beta actin or GAPD, each measured by qPCR. Untreated refers to combined cDNA from the saline treated animals. cDNA from each saline treated animal was also run against the pooled control cDNA.

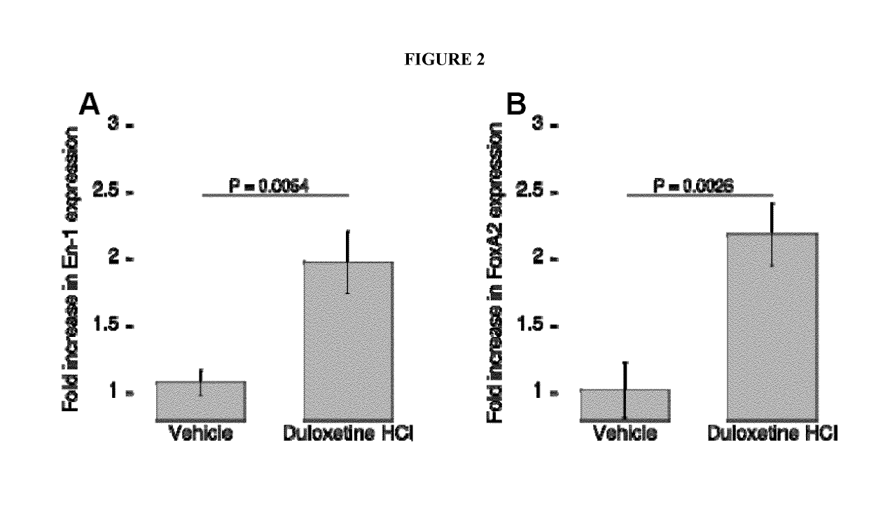

The results, as shown in FIG. 2, demonstrate that mice treated with duloxetine HCl had increased expression of En-1 mRNA in SNc as compared to mice treated with saline vehicle (1.98-fold increase±0.098 vs. 1.08-fold increase±0.23). Further, mice treated with duloxetine HCl had increased expression of FoxA2 as compared to mice treated with saline vehicle (2.19-fold increase±0.23 vs. 1.02-fold±0.21).

### Example 3

### Duloxetine HCl Exerts a Neuroprotective Effect on SN mDA Neurons

To confirm the neuroprotective effect of duloxetine HCl on mDA neurons, primary cultures of ventral midbrain cells of E12 CD1 mice were prepared. The embryonic ventral midbrains were removed and stored in HBSS on ice. The dissection time was less than 30-60 minutes. The ventral midbrain tissue was dissociated by removal of the HBSS and the addition of 1 ml of 0.05% trypsin-EDTA for 10 minutes at 37° C. During incubation, the tissue and the trypsin-EDTA solution was mixed by occasional careful swirling of the tube. The reaction was stopped, the trypsin aspirated, and 1 ml of 50% HBSS/FBS was added. The HBSS/FBS was then aspirated and the tissue was washed first in 1 ml HBSS and then in 1 ml E12 media (Dulbecco's modified Eagle's medium/F12 (Invitrogen)) containing 5% fetal bovine serum, IX N2 supplement A (Stem Cell Technologies), glucose (0.36%. Sigma), bovine serum albumin (0.25%, Invitrogen) and penicillin-streptomycin (Invitrogen). The tissue was triturated using an autoclaved glass pasteur pipette with narrowed opening and a stripettor. The cell suspension was centrifuged at 1000 rpm for 5 min, the supernatant aspirated, and the cells resuspended in E12 media. Twenty-five thousand cells per well were plated in 96 well PLO/laminin coated black-wall imaging plates (Costar) and incubated for 6 days without candidate compound treatment. Following this initial culture, cells were exposed to duloxetine HCl (10 μM) or DMSO vehicle for 24 hours. 6-hydroxydopamine hydrobromide (6-OHDA) (Sigma) was reconstituted in ascorbic acid and PBS and added at concentrations of 1-50 μM to the cultures. After 24 hours, the cultures were fixed using 4% paraformaldehyde. Cultures were stained for tyrosine hydroxylase, FoxA2 and Hoechst and visualized using alexa fluor secondary antibodies. Cultures were analyzed by high content analysis (using an InCell 2000 instrument). The percentage of FoxA2 positive dopaminergic neurons were documented. Data are presented as percent of control cells that have not been exposed to toxin.

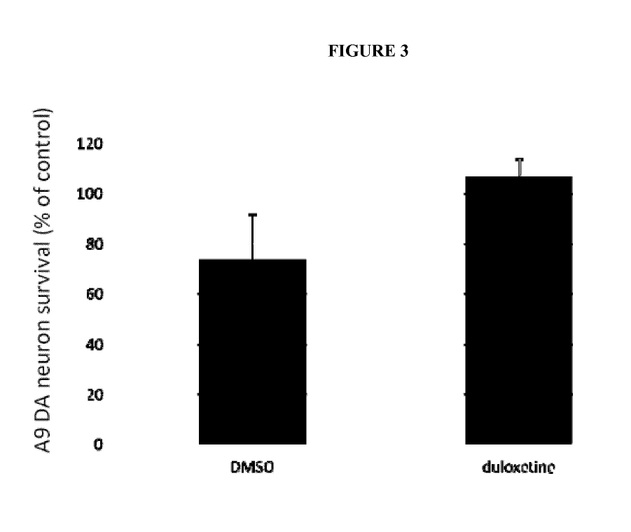

The results of the analysis are shown in FIG. 3 and confirm that pre-treatment of primary ventral midbrain cultures with duloxetine HCl as described above prior to the advent of PD-like toxicity, as induced by 6-OHDA, rescues substantia nigra dopaminergic neurons from toxicity as compared to vehicle- (DMSO) treated cultures. Indeed, survival as a percentage of control is 107%±7 for duloxetine HCl and only 74%±18 for DMSO-treated cultures.

### Example 3

### Use of Duloxetine for Prevention of Diseases Affecting mDA Neurons

A cohort of patients without diagnosed Major Depressive Disorder and with UPDRS scores of less than or equal to 10 will receive either 20 mg/day or 40 mg/day duloxetine HCl or placebo for 36 weeks. Scheduled in-clinic visits will be conducted at baseline and at weeks 4, 12, 24 and 36. At each in-clinic visit, a patient will be assessed and an updated UPDRS score calculated. Additionally, at each in-clinic visit, a PET scan will be conducted using CFT, labeled with either [¹⁸F] or [¹¹C], as an indicator of dopaminergic neurons. Therefore, altogether there will be 5 scheduled visits during the first 36 weeks. Unscheduled visits may be conducted at any time to assess a subject's need for additional anti-PD therapy, for safety reasons or for any other reason.

Based on the above randomization scheme, all subjects will receive active treatment (either 20 mg or 40 mg duloxetine HCl per day) for 36 weeks during this phase according to their original randomization allocation. Thus, subjects who receive 20 mg duloxetine during the first 36 weeks will continue to receive 20 mg during the second 36 weeks, subjects who receive 20 mg duloxetine during the first 36 weeks will continue to receive 20 mg during the second 36 weeks, and subjects who receive placebo during the first 36 weeks will receive either 20 mg or 40 mg duloxetine HCl during the second 36 weeks. The study blind will be maintained. No additional anti-PD therapy will be permitted during this phase. Scheduled in-clinic visits will be conducted every 6 weeks. Therefore, altogether there will be 6 visits during this phase at weeks 42, 48, 54, 60, 66 and 72. At each in-clinic visit, a patient will be assessed and an updated UPDRS score calculated. Additionally, at each in-clinic visit, a PET scan will be conducted using CFT as the ligand, and any mDA neuron loss will be calculated. Unscheduled visits may be conducted at any time to assess a subject's need for additional anti-PD therapy, for safety reasons or for any other reason.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for preventing or delaying the onset of a disease affecting mesenephalic dopaminergic neurons in a human suspected to be at risk for developing said disease comprising administering to said human a composition comprising duloxetine or a pharmaceutically acceptable salt thereof.

2. The method of claim 1, wherein the disease is Parkinson's Disease.

3. The method of claim 1, wherein the human has been identified as having a total UPDRS score of less than about 10.

4. The method of claim 3, wherein the human has been identified as having a total UPDRS score of 0.

5. The method of claim 1, wherein duloxetine is administered at a dose that results in the average total UPDRS score of the patient increasing less than 0.15 units per week.

6. The method of claim 5, wherein duloxetine is administered at a dose that results in an average total UPDRS score of the patient increases less than 0.05 units per week.

7. The method of claim 1, wherein the pharmaceutically acceptable salt of duloxetine is duloxetine HCl.

8. The method of claim 1, wherein the human has not been diagnosed with Major Depressive Disorder.

9. The method of claim 1, wherein the human is identified as having a 5-75% loss of midbrain dopaminergic neurons.

10. The method of claim 9, wherein the loss of dopaminergic neurons is less than about 65%.

11. The method of claim 9, wherein the loss of dopaminergic neurons is about 45% to about 65%.

12. (canceled)

13. The method of claim 1, wherein the human is administered about 40 to about 120 mg duloxetine per day.

14. The method of claim 1, wherein the human is administered about 40 to about 60 mg duloxetine per day.

15. A method of treatment of a patient suffering from a disease affecting mesenephalic dopaminergic neurons comprising administering to said patient a composition comprising duloxetine or a pharmaceutically acceptable salt thereof.

16. The method of claim 15, wherein the disease is Parkinson's Disease.

17. The method of claim 15, wherein the human has been identified as having a total UPDRS score of less than about 20.

18. The method of claim 15, wherein duloxetine is administered at a dose that results in the average total UPDRS score of the patient increasing less than 0.15 units per week.

19. The method of claim 18, wherein duloxetine is administered at a dose that results in an average total UPDRS score of the patient increases less than 0.05 units per week.

20. The method of claim 15, wherein the pharmaceutically acceptable salt of duloxetine is duloxetine HCl.

21. (canceled)

22. The method of claim 15, wherein the human is administered about 40 to about 120 mg duloxetine per day.

23. (canceled)
